(19) United States
(12) Patent Application Publication
(10) Pub. No.: US 2022/0310259 A1
(43) Pub. Date: Sep. 29, 2022

(54) System And Method For Determning Status Of Health Of Animals Arriving At A Feed Location

(71) Applicant: Intervet Inc., MADISON, NJ (US)

(72) Inventors: Robert S. SCHELLER, OVERLAND PARK, KS (US); Jason NICKELL, PARKVILLE, MO (US); Akshaya NATARAJ, WYNCOTE, PA (US)

(21) Appl. No.: 17/632,151

(22) PCT Filed: Aug. 3, 2020
(86) PCT No.: PCT/US20/44769

§ 371 Date: Feb. 1, 2022

Related U.S. Application Data
(60) Provisional application No. 62/883,937, filed on Aug. 7, 2019.

(51) Int. Cl. G16H 50/20; A61B 7/00; A61B 7/04

(52) U.S. Cl. CPC G16H 50/20; A61B 7/003; A61B 7/04

(57) ABSTRACT

A system and method are provided for screening and treating respiratory diseased animals such as livestock. Health measurements are taken of incoming animals to a location such as a feed yard. The measurements are used as inputs to an algorithm. Processing of the algorithm results in a prediction whether the animal is likely to become sick. A treatment decision is automatically generated by processing of the algorithm for display to a user. A “treat” decision may trigger a predetermined treatment protocol for the particular evaluated animal, while a “don't treat” decision allows the animal to be released.

### CROSS REFERENCE TO RELATED APPLICATIONS

This application is a national stage application under 35 U.S.C. 371 of PCT Application No. PCT/US20/44769 having an international filing date of Aug. 3, 2020, which designated the United States, which PCT application claimed the benefit of U.S. application Ser. No. 62/883,937, filed Aug. 7, 2019, entitled SYSTEM AND METHOD FOR DETERMINING STATUS OF HEALTH OF ANIMALS ARRIVING AT A FEED LOCATION. Both of which are incorporated by reference in their entirety.

### FIELD OF THE INVENTION

The invention generally relates to determining the health status of an animal, and optionally for, the diagnosis and treatment of diseased animals. More particularly the invention relates to a system and method for determining a health status of animals such as livestock arriving at a feed location. The invention is yet further directed to a system and method of health screening for animals arriving at the feed location, such as a feed yard, to determine the likelihood the animals have or may develop bovine respiratory disease (BRD) so that the animals may be timely diagnosed and treated.

### BACKGROUND OF THE INVENTION

It is a continual and complex challenge to maintain livestock at an optimum state of health at a location, such as a feed yard, where animals are held in a relatively tight quarters. Livestock frequently suffer from respiratory disease, commonly referred to as bovine respiratory disease (BRD) at feed yards for many reasons. BRD is highly contagious, and a relatively healthy group of incoming animals to a feed yard can quickly become infected considering the close proximity of the animals during the feeding operations. Livestock are intentionally metabolically stressed in a feed yard environment to promote rapid weight gain. Most livestock management protocols and nutrition practices strive to maximize daily weight gain. However, the accelerated weight gain experience by livestock may degrade the ability of animals to mount natural compensatory responses to the environmental stresses placed upon the livestock. The metabolic challenges of weight gain, the exposure to infected animals, being placed within an unfamiliar environment and generally crowded conditions all contribute to stresses placed upon each animal's immune system.

There are a number of treatment approaches that have been undertaken to timely and effectively identify potentially sick and diseased animals who have been introduced to a feeding facility. One common treatment protocol is to treat all incoming animals to a feeding facility with an initial round of antibiotics as a preventative measure. While uniform administration of an antibiotic has the advantage of reducing occurrences of acute BRD for the incoming group of animals, there are of course disadvantages to include cost and the general undesirability of administrating antibiotics to healthy animals.

There are also a number of diagnostic protocols available to caregivers in determining the particular health status of an animal arriving at the feeding location. Respiratory and cardiovascular auscultation analysis is known for both livestock and humans. To make a diagnosis of an element based upon auscultated sounds, extensive empirical knowledge of various and diverse forms of auscultated sounds is necessary. In the past, auscultation analysis was in large part dependent upon the trained ear of the caregiver to discern abnormal auscultations since measured auscultation data may not have been available. More recently, with the development of electronic stethoscopes, auscultated sounds could be recorded in digital form. Software programs were developed to analyze the data to determine characteristics of the data which would indicate the severity of a disease condition such as BRD.

Despite the number of treatment protocols developed for treatment of BRD and other respiratory conditions of livestock in a feed yard environment, there are still significant shortcomings in treatment options that either sacrifice cost parameters by overtreatment, or under treat animals resulting in later economic losses by increased numbers of mortalities or acute disease occurrences.

Accordingly, there is still a need for an automated process or method that can result in a timely and economically viable diagnosis of BRD for large groups of animals located in environment such as feed yard facilities. There is also a need to provide the process or method that can be easily integrated within existing feed yard facilities without the need for significant capital expenditures on new equipment or significant data processing modifications. There is also a need to provide a process or method that can provide a caregiver with a nearly instantaneous indication as to whether a particular animal may require immediate treatment or whether the animal is healthy enough that it may be introduced into the livestock population within the feed facility without treatment.

These needs and others are met with the invention disclosed herein, as set forth in the following description and claims.

### SUMMARY OF THE INVENTION

In accordance with the present invention, in one or more preferred embodiments, a system and method are provided for screening and treating respiratory diseased animals such as livestock.

In more general terms the invention, in various embodiments, provides an indication of a general health status of an animal; however, the invention does not require a diagnosis or treatment recommendation. Accordingly, it shall be understood that there is utilitarian value in determining a general health status of the animal to trigger other subsequent decisions regarding the animal. These other decisions may include diagnosis or treatment, but may also include decisions such as to whether to transport an animal to another location, to provide a different feeding protocol for the animal, to provide a different environment for the animal such as removal of the animal from a feed yard, and other decisions that involve the disposition of the animal.

According to another embodiment of the invention, it includes a data processing system that is directed to the method of screening and treating respiratory diseased animal.

According to one or more preferred embodiments, upon arrival of an animal to the feedlot or other location, measurements are taken of each incoming animal. These measurements are inputs into one or more algorithms which are used to provide a general health status of the animal, and to also optionally predict whether the animal is likely to become sick, to include whether the animal may already show symptoms or indications of being sick. The primary disease condition addressed by the present invention is BRD; however, principles of the invention are also applicable to other respiratory conditions of the animals being evaluated.

Upon arrival, measurements taken of an animal include the respiratory rate, heart rate, rectal temperature, and body weight. These measurements are recorded and stored in a data processing system of the invention. For each animal evaluated upon arrival, the measurements are used as inputs to one or more algorithms which in turn, provide an indication of the health status of the animal. The rectal temperature and body weight measurements can be considered direct inputs to the algorithms. The data transferred from corresponding field devices such as digital thermometers and weigh scales are provided in digital forms that do not require additional signal processing. However for the respiration and heart rate data, the data obtained is by auscultation, thus the data is in the form of raw sound data (such as a .WAV format) that must undergo some processing to digitize the data in a form that can be used in the algorithm. Signal processing may include, for example, harmonic extraction and Fourier transform analyses to obtain appropriate signal amplitudes representative of the actual respiratory and heart rates.

According to one preferred embodiment of a method of the invention, a determination is made as to a health status of the animal to trigger a subsequent recommendation or decision. According to a particular preferred embodiment, the decision is whether to treat an animal or not treat the animal based upon use of a gradient boosted tree algorithm in which execution of the algorithm generates a “treat” or “don't treat” decision. For example, a “treat” decision may trigger a predetermined treatment protocol for the particular evaluated animal, while a “don't treat” decision calls for releasing the animal to the feedlot population without any treatment.

One general characteristic of the algorithm is that the decision generated for treat/don't treat is weighted more heavily for treatment as opposed to no treatment. Another general characteristic of the algorithm is that the algorithm is more heavily influenced or “trained” based upon cases of morbidity that are identified within the first 60 days on arrival. This 60 day window provides a reliable indication as to both active BRD for animals on arrival as well as transmitted cases of BRD while the animal is retained within the feedlot. In other words, this window is effective to capture BRD diseased animals that were sick on arrival to the feedlot as well as animals that contract the disease from other animals that may have been sick upon arrival.

Gradient boosting is known as a machine learning technique for both classification and regression problems. The execution of a boosted tree algorithm provides a prediction model in the form of decision trees. The model defines the problem to be solved in which the decision trees provide the framework for an incremental analysis of the problem but ultimately allowing optimization of the analysis of predetermined parameters making up the decision trees, referred to as decision “stumps” or binary recursive partitions.

Stated in another way, the machine learning technique of the invention enables a programmed computer processor to “learn” or otherwise optimize a solution to a defined problem, without the processor having to be explicitly programmed to optimize the solution despite wide variances in input data as well as wide variances in data patterns or trends. Accordingly, the gradient boosting algorithm of the invention results in a computer processor automatically providing an optimal solution, such as the treat/don't treat decision.

According to one method of the invention, in one or more preferred embodiments, the gradient —boosted tree algorithm (hereinafter the “algorithm”) generates a decision to treat/not treat based on a majority decision of the results of the algorithm where a “treat” decision is weighted more heavily than the “do not treat” decision, as mentioned.

The recorded breathing sounds may be expressed as a selected summary of features (h1, . . . h10) wherein these 10 features have been shown to be an adequate parametrization of the sample sound data.

As discussed in further detail below, another feature of the invention is the use of a digital electronic stethoscope for auscultation in which the stethoscope has a plurality of audio sensors. A recorded sound event for use in the algorithm may be an average or pre-selected synthesis of raw sound signal data recorded by the audio sensors. Further, the recorded sounds have values extracted from the corresponding raw signal data recorded in which one or more bandpass filters are applied to capture breathing attributed to acute, chronic disease condition frequencies, and ambient noise frequencies. For example, 5 bandpass filters could be applied in which the filters are arranged for recorded sounds representative of the disease condition and ambient noise frequencies as follows: (1) an IIR Butterworth filter with FS=1000 Hz and FC=400 Hz applied to capture breathing attributed to chronic level of disease-severity (2) an IIR Butterworth filter with FS=1000 Hz and FC=80 Hz applied to capture breathing attributed to an acute level of disease severity; (3) An IIR Butterworth filter with FS=1000 Hz and FC=180 Hz is applied to capture quiet ambient noise levels in the recording useful for normalization; (4) an IIR Butterworth filter with FS=1000 Hz and FC=115 Hz is applied to capture medium ambient noise levels in the recording useful for normalization; and (5) an IIR Butterworth filter with FS=1000 Hz and FC=90 Hz Is applied to capture low ambient noise levels in the recording useful for normalization. It should be understood that these five bandpass filter conditions are provided as examples. Accordingly, different bandpass filter arrays can be applied in response to any desired disease condition or ambient noise frequencies that may be presented.

The recorded respiratory rate in may be expressed as (rr). The recorded heart rate in the algorithm may be expresses as (hr). The recorded body weight (bw) is used in the algorithm as a proxy for age. The rectal temperature may be expressed as (rt). Rectal temperature, which is an indication of current disease, is not required to be a direct algorithm input, but may be a direct algorithm input when data is available.

Some additional inputs to the algorithm include expressions of the recorded data to include the following: (a) a cardio-pulmonary ratio (cpr), calculated as

$$( \frac{hr percentile}{rr percentile} )$$

is a ratio that represents the magnitude of stress experienced by cattle on arrival. The greater the value of the ratio, the greater the stress experienced by the animal; (b) a cardiac breath gap (cbg), calculated as (hr−rr). (c) a cardiac index (ci), calculated as

$$( \frac{hr percentile}{bw percentile} ) ,$$

that is a relationship between heart performance and cattle size; and (d) a cardiac max (cm), calculated as

$$( \frac{hr}{75 th percentile hr ( based on bw and rt )} ) ,$$

is an estimation of how efficiently the animal is consuming oxygen on arrival

One advantage to providing multiple inputs to the algorithm of the present invention, as opposed the prior art that has fewer inputs and does provide input combinations of the invention as described herein, is that the prior art algorithms or logic are more prone to generating incorrect decisions or conclusions in which one input factor or variable may overly influence the outcome of the algorithm.

According to preferred embodiments of the invention, there are a number of architecture models that may be adopted. Four of these models may include the following: (1) a model including both bw and rt; (2) a model including bw and not rt; (3) a model including rt and not bw; and (4) a model including neither. Though inputs may be omitted based on which model is applied, the tree-generation process is the same for each selected architecture model.

The gradient boosted tree algorithm of the invention can be interpreted as a series of conditional statements involving a subset of the number of inputs. The gradient boosted tree algorithm may be represented by exemplary model architecture that comprises a plurality of decision stumps, or a binary recursive partitions.

For example, at node Jᵢ, an instance is passed to the left if it satisfies the condition relating to input variable Xᵢ, and right otherwise.

Continuous inputs fulfill mathematical operators (<, >, ≥, ≤) in relation to a threshold tᵢ, whereas nominal inputs must simply be members of an explicit list of values (ϵ [a, b, . . . , z]). Repeated splits on the same input are allowed. According to one preferred embodiment of the algorithm, the inputs are all continuous and comprise the data captured that is common to all animals being evaluated, that is, the data captured for rectal temperature, body weight, respiration rate and heart rate. According to another embodiment of the algorithm, it may further incorporate nominal inputs, that is, additional information about other preselected or predefined categories of the animals. Once example of a nominal input is classifying cattle based on breed. Accordingly, a treatment decision could be generated based on breed-particular conditions that could vary between breeds.

A sequence of nested stumps collectively forms a tree, also known as a weak learner or classifier. Values for thresholds t are chosen by optimizing a “split function”, which chooses the best input variable and the best value for that input variable that result in the largest reduction in “cost”. There is also a penalty for misclassifying, which is represented empirically by a mathematical function “Cost” may be defined as the error or deviation in a predicted output from an actual output. A best input value is chosen based on the largest reduction in cost or smallest mean error calculated when optimizing a split function. For example, an optimal statistical measure of distribution may be chosen, such as a Gini Index, which is one of many that can be used to measure purity of groups of data created by a split function.

The gradient boosting tree algorithm of the invention may be optimized by executing sequentially created new classifiers and repeatedly applying these classifiers to re-weighted versions of training data. The training data may comprise a percentage of previously recorded data over a period of time.

After each boosting iteration, or creation of a new classifier, misclassified instances of the training data have their weights increased, and correctly classified instances have their weights decreased. Hence, each successive classifier focuses on instances that have been hard to classify in the previous steps. By executing the gradient boosting tree algorithm of the invention, this methodology minimizes the number of instances of “false positive” conclusions, for example, assigning a sick animal with the decision to not treat upon arrival to the feed facility.

According to the invention, each model generated m ϵ (1, 2, . . . , M) is added sequentially until no further improvements can be made, that is, no further reductions in cost can be achieved or no smaller errors are capable of being computed. New models are created that predict the residuals or error of prior models, and then added together to make a subsequent improved model. After M iterations, the predictions of each model/weak learner are combined into a final prediction based on a weighted majority vote In other words, each instance is passed to each tree/weak learner and classified as either a treat/don't treat decision. The final decision or majority vote of the overall classifier is the total weighted counts of the individual treat/don't treat learners.

According to one aspect of the invention, the algorithm of the invention can be expressed as a base mathematical function. The mathematical function can be defined as a gradient boosted tree algorithm as follows:

F₀(x)=arg min γ Σᵢ₌₁ⁿL(yᵢ, γ) in which the function classifies a decision to treat or not treat an animal, and where:

γ represents the specific combination of inputs that arrive at the decision, and

Σ L represents the misclassification error, or number of falsely estimated instances i in the training set when compared to the true label yᵢ. The invention seeks to minimize the classification error.

The gradient-boosted algorithm improves on classifier F₀ by constructing a new classifier h(x), that adds an estimator to provide a better model i.e. Fₘ₊₁(x)=Fₘ(x)+hₘ(x). A perfect h(x) would imply that Fₘ(x)+h(x)=y, the true label. Therefore gradient-boosting will fit h(x) to the residual y−Fₘ(x). For m ϵ (1, 2, . . . , M), compute a pseudo-residual (

$${r}_{im} = - {[ \frac{\partial L ( {y}_{i} , F ( {x}_{i} ) )}{\partial F ( {x}_{i} )} ]}_{F ( x ) = {F}_{m - 1} ( x )}$$

for each instance i. The pseudo-residual rᵢₘ is the numerical representation of the degree to which this instance was misclassified. Next, fit a classifier hₘ(x) to the pseudo-residuals i.e. train it using the training set (xᵢ, rᵢₘ) rather than (xᵢ, yᵢ). Capture the effects of the misclassified data via the multiplier γₘ, which is computed by solving the optimization problem:

γₘ =arg min γ Σᵢ₌₁ⁿL(yᵢ, Fₘ₋₁(Xᵢ)+γ^{h}ₘ(xᵢ)).

Next, Update the classifier as Fₘ(x)=Fₘ₋₁(x)+γₘhₘ(x), which represents a gradient-boosted tree, as the misclassified instances have had their weights increased by γₘ. The final prediction or output F_{M}(x), is computed as the majority vote of the individual predictions from each Fₘ(x).

Below is one schematic representation of an exemplary gradient boosted tree F_{M1} in which the tree comprises a plurality of decision stumps. In this example, the algorithm is executed and the “vote” or outcome of the executed algorithm results in a “don't treat” decision.

Below is another schematic representation of an exemplary gradient boosted tree F_{M2} in which the tree again comprises a plurality of decision stumps. In this example, the algorithm is executed and the “vote” or outcome of the executed algorithm results in a “ treat” decision.

In summary, a number of classifiers or individual trees are executed. The majority vote or decision to treat or not to treat an animal can be determined by the majority treat/don't treat conclusion/decision from each decision tree. However, in accordance with the present invention, a treat conclusion/decision from an executed decision tree is preferably weighted more heavily than a don't treat conclusion/decision, since it is desirable to prevent missing a potential instance of treating a sick animal. It is also contemplated within the invention that any executed decision tree with a treat conclusion/decision can result in an output to a user instructing treatment of the animal.

The iterative computations conducted in the boosting tree algorithm of the invention creates shallow decision trees (i.e. a small J), and sources the misclassification error of the previous trees to sequentially build a strong ensemble, or final majority vote, of weak learners/classifiers. This differs from a comparable approach known as “random forest”, which consists of creating many deep but de-correlated trees in parallel. The number of iterations/trees M is typically larger, though each is still based on a random subset of inputs/features p and grown to a maximum depth J, with a final prediction based on a majority vote.

The boosted tree algorithm of the invention is advantageous because it is easy to interpret, performs automatic input-variable selection, scales well to large data sets and is relatively robust to outliers.

By the foregoing, it should be understood that the algorithm of the invention is not a static mathematical equation that results in calculation of a decision to treat or not treat based on a single computation of an equation. On the contrary, the algorithm of the invention may also be considered or defined as a plurality of sequential logic computations in which “machine learning” characteristics of a boosted tree architecture optimizes a decision to treat or not treat a particular animal being evaluated.

One preferred way to obtain input for measurement data is by stethoscopic evaluation of an animal. Collection of heart and lung sounds is preferably obtained by a digital/electronic stethoscope. Optionally, it may be desirable to select a stethoscope that is capable of expressing sounds in the form of a visible spectrogram so that the user may confirm heart and lung sounds are being obtained. Data collected from the stethoscope is stored and then used within the algorithm of the invention by a computer processor that executes computer coded instructions corresponding to the algorithm as selectively programmed.

The specific placement of the stethoscope can be determined by a caregiver, it being known that one manner to obtain heart and lung sounds is to place the digital stethoscope a few inches above the right elbow of the animal thereby placing the stethoscope over the right apical lobe. Similarly, sounds can be gathered on the left side a few inches above the left elbow thereby placing the stethoscope over the left cardiac lobe.

According to one aspect of the invention, one preferred device for capturing auscultated sounds from an animal includes a digital stethoscope incorporating a chest piece that communicates wirelessly with a computer processor to receive and store the recoded data. The selected computer processor applies the algorithm to the recorded data, and a tangible output is produced that provides an indication of whether to treat or not to treat the animal. This output may be communicated to one or more mobile communication devices that are held by the caregiver thereby providing a caregiver a nearly instantaneous decision as to sorting of the animal at the arrival location and whether the animal requires treatment. Typically, as an animal enters a feeder, the animal is processed within a confined chute. Based upon experimentation, it is been found that once the stethoscope records animal data, the algorithm can be run on a selective computing device and an output to the caregiver can be provided in less than about 10 seconds. If treatment is required, the animal can be held within the chute to receive treatment that corresponds to the treatment decision, such as one or more inoculations.

In addition to the treat/don't treat decision, other indications can be generated for the caregiver which may include a message or report that summarizes the perceived health of the animal as judged by the algorithm applied to the recorded heart and lung sounds. One example may include a message produced on a user interface of a computing device that shows the raw recorded data along with a recommendation for treatment such as dosage for a particular medication. Other information can be displayed about the animal that was evaluated, such as historical treatment information which may be used to determine the proper current treatment if execution of the algorithm results in the treat decision.

According to another aspect of the invention, the digital stethoscope may be more particularly in the form of a wireless audio digital recording unit that incorporates the use of a multi-channel microphone. The multi-channel microphone may have a plurality of sensors in which the sensor readings can be averaged or normalized thereby providing a more accurate way to record data. The recording unit may include a curved shape paddle that is designed to conform to match the curvature of the chest area of the particular animal which the devices to be used. The recording unit can be secured to an extension arm held by the caregiver enabling the caregiver to be positioned in a safe distance from the animal within the chute.

Considering the foregoing, according to one particular aspect of the invention, it may be considered a method for screening and treating an animal utilizing recorded auscultations and other recorded data of the animal including body weight and rectal temperature.

According to another aspect of the invention, it may be considered an integrated system for determining a health status of an animal utilizing the recorded auscultations and other recorded data. The integrated system includes one or more data processing devices, a number of field devices that communicate with the data processing device(s) for gathering extensive data regarding the incoming animals and to provide predictive health observations to a caregiver. The field devices at least include a digital audio recording unit for recording auscultations. Other field devices may include weigh scales, temperature probes, RFID readers, and other diagnostic equipment. A desired communication protocol may be adopted for communication between the field devices and the data processing device(s) such as wireless communication protocol.

According to yet another aspect of the invention, it may be considered a data processing system for automatically generating a decision whether to treat an animal by use of an algorithm with inputs to the algorithm comprising the recorded auscultations and other recorded data, and subsequently providing a viewable output to a user that conveys the decision.

According to one particular preferred embodiment of the invention, it may be expressed as a system for determining a health status of an animal upon arrival at a location, comprising: a computer processor for receiving and storing biometric data; at least one recording device for capturing and recording biometric data from the animal, said device communicating with said computer processor, said device including at least an audio recorder that records the biometric data obtained by auscultated heart and lung sounds of the animal; computer coded instructions executed by said computer processor including at least one algorithm for determining the health status; a user interface associated with the computer processor for displaying information obtained by execution of said computer processor; and wherein said algorithm includes a gradient boosted tree algorithm comprising input variables obtained from the recorded biometric data and corresponding to the recorded heart and lung sounds.

Further according to this first particular preferred embodiment, a number of additional optional features may be included. These optional features may be provided alone or in combination with one another as follows:
(1) wherein said input variables further include at least one of a body weight of the animal and a rectal temperature of the animal;
(2) wherein said algorithm incorporates the biometric data recorded as converted to numerical form for inputs to the algorithm, and in which raw signal dated recorded of the auscultated heart and lung sounds undergo bandpass filtering;
(3) wherein said input variables include a heart rate;
(4) wherein said input variables include a respiration rate;
(5) wherein said algorithm includes additional inputs including arithmetic expressions of the recorded biometric data, said expressions including at least one of a cardiopulmonary ratio, a cardiac breath gap, a cardiac index, and a cardiac max;
(6) wherein another user interface is provided and associated with the computer processor for displaying a health status of the animal, said health status including a likelihood the animal may develop BRD;
(7) wherein said displayed information includes a user treatment decision for the animal;
(8) wherein said at least one algorithm is a gradient boosted tree algorithm expressed by a function:

F₀(x)=arg min γ Σᵢ₌₁ⁿL(yᵢ, γ);
wherein the function classifies the treatment decision to treat or not treat an animal; wherein γ represents a specific combination of inputs that arrive at the treatment decision; and
wherein Σ L represents a misclassification error or number of falsely estimated instances i in a training set when compared to a true label yᵢ;
(9) wherein said specific combination of inputs y includes at least one of:
   (a) a cardio-pulmonary ratio (cpr), calculated as

$$( \frac{hr percentile}{rr percentile} ) ,$$

said cpr ratio representing a magnitude of stress experienced by the animal upon arrival at the location;
(b) a cardiac breath gap (cbg), calculated as (heart rate−respiration rate);
(c) a cardiac index (ci), calculated as

$$( \frac{hr percentile}{bw percentile} ) ,$$

said ci index representing a relationship between heart performance and size of the animal; and
(d) a cardiac max (cm), calculated as

$$( \frac{hr}{75 th percentile hr ( based on bw and rt )} ) ,$$

said cm representing an estimation of how efficiently the animal is consuming oxygen upon arrival at the location;
(10) wherein execution of said at least one algorithm by said computer processor includes a plurality of decision stumps and a generated outcome resulting in a treatment decision;
(11) wherein said treatment decision is a treat decision;
(12) wherein said treatment decision is a don't treat decision; and
(13) wherein execution of said at least one algorithm includes conducting iterative computations to arrive at the treatment decision.

According to another particular preferred embodiment of the invention, it may be expressed as a method for determining a health status of an animal upon arrival at a location, comprising:

providing a computer processor for receiving and storing biometric data; providing at least one recording device; recording raw biometric data from the animal by use of at least an audio recorder that records heart and lung sounds; converting the raw biometric data recorded to manipulated heart and lung sound data; providing computer coded instructions executed by the computer processor including at least one algorithm for determining a health status of the animal; executing the algorithm with input variables corresponding to the manipulated heart and lung sound data; generating a user interface associated with the computer processor for displaying to a user a health status of the animal; and wherein said algorithm includes a gradient boosted tree algorithm comprising input variables corresponding to the manipulated heart and lung sound data of the animal.

Further according to this second particular preferred embodiment, a number of additional optional features may be included. These optional features may be provided alone or in combination with one another as follows:
(1) wherein said at least one algorithm further includes computer coded instructions for determining whether the animal should receive treatment; and said user interface further includes a treatment decision for the animal;
(2) wherein said input variables further include at least one of a body weight of the animal and a rectal temperature of the animal;
(3) wherein said manipulated heart and lung sound data includes the raw biometric data that undergoes bandpass filtering;
(4) wherein said manipulated heart and lung sound data includes a recorded heart rate;
(5) wherein said manipulated heart and lung sound data includes a respiration rate and selected recorded frequencies and amplitudes of recorded lung sounds;
(6) wherein said algorithm includes additional inputs including arithmetic expressions of recorded heart and lung sound data, said expressions including at least one of a cardiopulmonary ratio, a cardiac breath gap, a cardiac index, and a cardiac max;
(7) wherein the method further includes generating a user interface associated with the computer processor wherein said health status including a likelihood the animal may develop BRD;
(8) wherein said health status includes a user treatment decision for the animal;
(9) wherein said at least one algorithm is a gradient boosted tree algorithm expressed by a function:

F₀(x)=arg min γ Σᵢ₌₁ⁿL(yᵢ, γ);
wherein the function classifies the treatment decision to treat or not treat an animal; wherein γ represents a specific combination of inputs that arrive at the treatment decision; and
wherein Σ L represents a misclassification error or number of falsely estimated instances i in a training set when compared to a true label yᵢ;
(10) wherein said specific combination of inputs γ includes at least one of:
   (a) a cardio-pulmonary ratio (cpr), calculated as

$$( \frac{hr percentile}{rr percentile} ) ,$$

said cpr ratio representing a magnitude of stress experienced by the animal upon arrival at the location;
(b) a cardiac breath gap (cbg), calculated as (heart rate−respiration rate);
(c) a cardiac index (ci), calculated as

$$( \frac{hr percentile}{bw percentile} ) ,$$

said ci index representing a relationship between heart performance and size of the animal; and
(d) a cardiac max (cm), calculated as

$$( \frac{h r}{75 th percentile hr ( based on bw and rt )} ) ,$$

said cm representing an estimation of how efficiently the animal is consuming oxygen upon arrival at the location;
(11) wherein execution of said at least one algorithm by said computer processor includes a plurality of decision stumps and a generated outcome resulting in a treatment decision;
(12) wherein said treatment decision is a treat decision;
(13) wherein said treatment decision is a don't treat decision; and
(14) wherein execution of said at least one algorithm includes conducting iterative computations to arrive at the treatment decision.

According to yet another particular preferred embodiment of the invention, it may be expressed as a non-transitory computer-readable medium containing computer executable instructions, wherein, when executed by a computer processor, the instructions cause the computer processor to execute a method to determine a health status of an animal the computer-readable instructions comprising: instructions to receive and store data corresponding to recorded heart and lung sounds; instructions to convert the recorded heart and lung sounds to manipulated heart and lung sound data; instructions to execute an algorithm to determine the health status of the animal, wherein the algorithm comprises input variables corresponding to the manipulated heart and lung sound data; instructions to generate a user interface associated with the computer processor for displaying to a user the health status of the animal, wherein said health status includes technical recommendations for further evaluating whether the health status requires further analysis; and wherein said algorithm includes a gradient boosted tree algorithm comprising input variables corresponding to the manipulated heart and lung sound data of the animal.

Further according to this third particular preferred embodiment, a number of additional optional features may be included. These optional features may be provided alone or in combination with one another as follows:
(1) wherein said instructions to execute the algorithm further include instructions to determine whether the animal should receive treatment; and said instructions to generate the user interface further include instructions to display to the user a treatment decision for the animal;
(2) wherein said health status includes a user treatment decision for the animal;
(3) wherein said at least one algorithm is a gradient boosted tree algorithm expressed by a function:

F₀(x)=arg min γ Σᵢ₌₁ⁿL(yᵢ, γ);
wherein the function classifies the treatment decision to treat or not treat an animal;
wherein γ represents a specific combination of inputs that arrive at the treatment decision; and
wherein Σ L represents a misclassification error or number of falsely estimated instances i in a training set when compared to a true label yᵢ;
(4) wherein said specific combination of inputs y includes at least one of:
   (a) a cardio-pulmonary ratio (cpr), calculated as

$$( \frac{hr percentile}{rr percentile} ) ,$$

said cpr ratio representing a magnitude of stress experienced by the animal upon arrival at the location;
(b) a cardiac breath gap (cbg), calculated as (heart rate−respiration rate);
(c) a cardiac index (ci), calculated as

$$( \frac{hr percentile}{bw percentile} ) ,$$

said ci index representing a relationship between heart performance and size of the animal; and
(d) a cardiac max (cm), calculated

$$( \frac{h r}{75 th percentile hr ( based on bw and rt )} ) ,$$

as said cm representing an estimation of how efficiently the animal is consuming oxygen upon arrival at the location;
(5) wherein execution of said at least one algorithm by said computer processor includes a plurality of decision stumps and a generated outcome resulting in a treatment decision;
(6) wherein said treatment decision is a treat decision;
(7) wherein said treatment decision is a don't treat decision; and
(8) wherein execution of said at least one algorithm includes conducting iterative computations to arrive at the treatment decision.

It should be understood that while embodiments of the invention are particularly suited for animals such as cattle arriving at a feedlot, the feedlot environment for cattle is just one particular environment in which the invention can be applied. Other examples in which the invention applies includes, but is not limited to, determining a health status of, and optionally diagnosis and treatment, for swine, sheep, horses, bison, and other domesticated animals. Further, while the arrival location is described as a feedlot or feed yard, the invention is not limited to any particular arrival location.

Other features and attributes of the invention will become apparent from the following detailed description taken in conjunction with the exemplary figures.

### BRIEF DESCRIPTION OF THE DRAWINGS

FIG. 1 is an example user interface showing information for entering data regarding an animal to be added to the data processing system in order that a health status can be obtained and optionally, to obtain a treatment decision that can be made once data is recorded; this figure also shows selected information about animals who have already been processed, sorted by lots within a feed yard;

FIG. 2 is another example user interface that displays a health record including a treat/no treat decision for an animal that has been processed. The user interface also shows additional selected information about the animal, information about the feed yard, and notes that may be added by the caregiver;

FIG. 3 is a perspective view of an example wireless audio digital recording unit that can be used to obtain and record auscultations for data entry;

FIG. 4 is a bottom plan view of a recording paddle of the audio digital recording unit showing a plurality of audio sensors; and

FIG. 5 is a block diagram showing a system of the invention including a number of data processing devices and field devices enabling recorded animal data to be processed and subsequently communicated within a communications network for treating selected animals.

### DETAILED DESCRIPTION

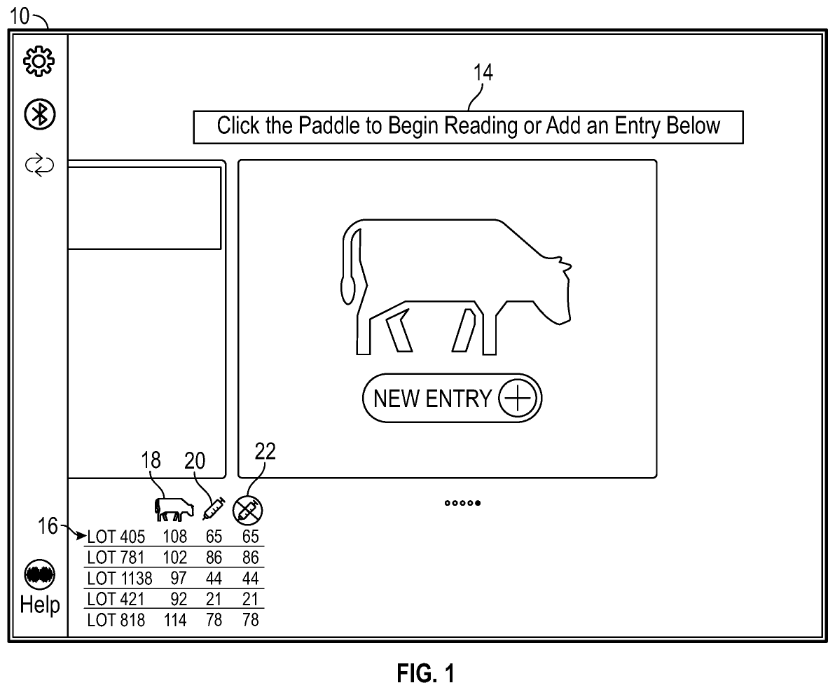

FIG. 1 is an example user interface 10 associated with entering, recording and processing data of an animal on arrival to a location such as a feed yard. More specifically, this figure provides a schematic depiction or representation of an animal for which a new data record 12 is to be created for the animal as it is received to the location. The animal may be specifically identified, for example, by use of an RFID tag that is associated with the animal. The tag may contain detailed information about the animal to include its prior health history and other data. Entering the tag data is one example of how an animal may be initially set up and recorded in a data processing system of the invention enabling subsequent tracking and treatment.

Once the user completes entry of new information about the animal record to be created, or if information about the animal is has been automatically uploaded (such as by RFID tag data), this figure also shows a function for the user to begin reading biometric data for the animal, shown at block 14. A digital recording unit such as an electronic stethoscope can be used to record auscultation data. Accordingly, as detailed further below, the digital recording unit communicating with a data processor of the associated computer processing system of the invention enables the upload of the auscultation data.

FIG. 1 also shows information about the number and location of animals within the feed yard, shown as Lot numbers 16 with the corresponding number of animals 18 found in each lot at that time, along with a count of whether the animals have been treated or not treated. The treated animals are shown with a syringe symbol 20 while those animals not treated are shown with the syringe symbol crossed out 22.

FIG. 1 is an example user interface with certain functional attributes that enable a user to create a new record for an animal arriving at the location and to begin entering a biometric data, it being understood that the particular user interface can be alternatively arranged in a manner that is most convenient for visualization by the user. In addition to auscultation data, a user may also enter information regarding the body weight and rectal temperature of the animal. Each of these additional data elements can be automatically uploaded by field devices that communicate with the data processor responsible for handling algorithm calculations. For example, a weigh scale and digital rectal thermometer can be co-located within a chute which holds the animal while recording of auscultations are conducted.

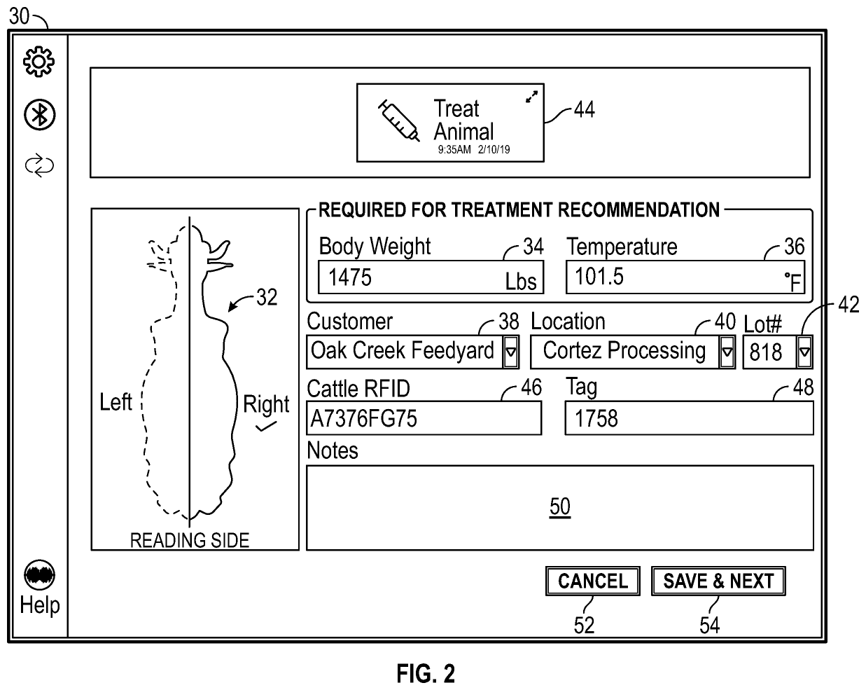

FIG. 2 provides another example of a user interface 30 showing information recorded about the animal. More specifically, this figure shows a schematic representation of the animal 32 and which side of the animal was used for recording heart and lung sounds. In this example, the right side of the animal was contacted by a1 recording unit. The body weight 34 and temperature 36 are also displayed, along with the feed yard owner/operator 38, location 40, and lot number 42 where the animal is located.

FIG. 2 also displays the treatment decision that is generated once the algorithm is processed with the recorded data of the animal. As shown, the treatment decision 44 is depicted with a syringe symbol along with a date and time in which the treatment decision was created. The treatment decision is generated within a very short time period after the recorded data is entered so that the animal can be treated while being held in the chute or other confined location in which the recorded data is obtained from the animal.

Further information shown in the sample user interface of FIG. 2 includes an RFID identification number or code 46 and an optional tag identifier 48 that may be used for identification purposes only within the feed yard. Another optional feature of this sample user interface is a notes section 50 that may be populated with special instructions for the care of the animal or any other notes a caregiver may wish to add. FIG. 2 also illustrates standard navigation features shown as a cancel function 52 and a save/next function 54 enabling the user to navigate between user interfaces as desired.

Although FIG. 2 specifically illustrates an example of a treatment decision, it shall be understood that the invention does not require a decision to be generated only regarding a diagnosis and/or treatment. The invention has broader applicability and can be used to generate other decisions such as whether to transport an animal to another location, to provide a different feeding protocol for the animal, or to provide a different environment for the animal such as removal of the animal from a feed yard where the animal may experience a higher level of stress. Specifically, for example, a decision can be made whether to transport an animal to another location if the animal exhibits recorded data indicative that the animal is unduly stressed or potentially sick. This decision to transport or not transport is not made as a result of any particular diagnosis or treatment decision, but rather, based on other criteria that indicates the health status of the animal is such that the animal should be further evaluated and/or isolated as a precaution.

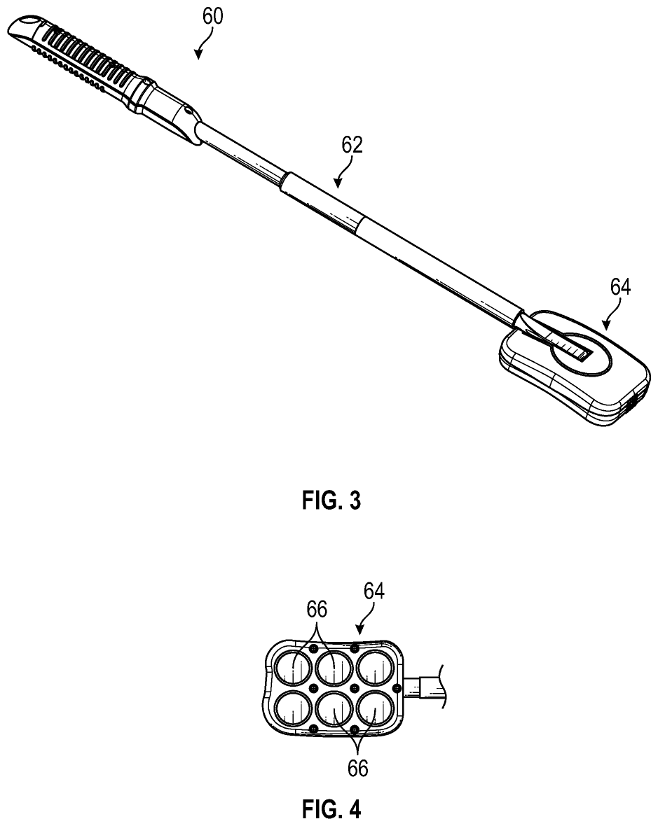

FIG. 3 illustrates one example of an audio digital recording unit 60 that can be used to gather auscultation data including heart and lung sounds. The recording unit is capable of recording and transmitting the data wirelessly to a remote computing device. Alternatively, the unit itself may have its own microprocessor, memory, software/firmware and database(s) for manipulation of data to record and generate a tangible output for the user to receive and further manipulate on a another computing device. The unit 60 is depicted as having an arm or extension 62, a handle 64, and a paddle 64 that houses audio sensors for recording sounds.

Referring also to FIG. 4, the panel may incorporate a plurality of audio sensors 66. The use of redundant audio sensors as opposed to a single sensor may provide more reliable data for use in the algorithm. A single audio output from the group of sensors may be achieved by computing mean averages of the recorded data and then generating a single audio signal reflective of the computed averages.

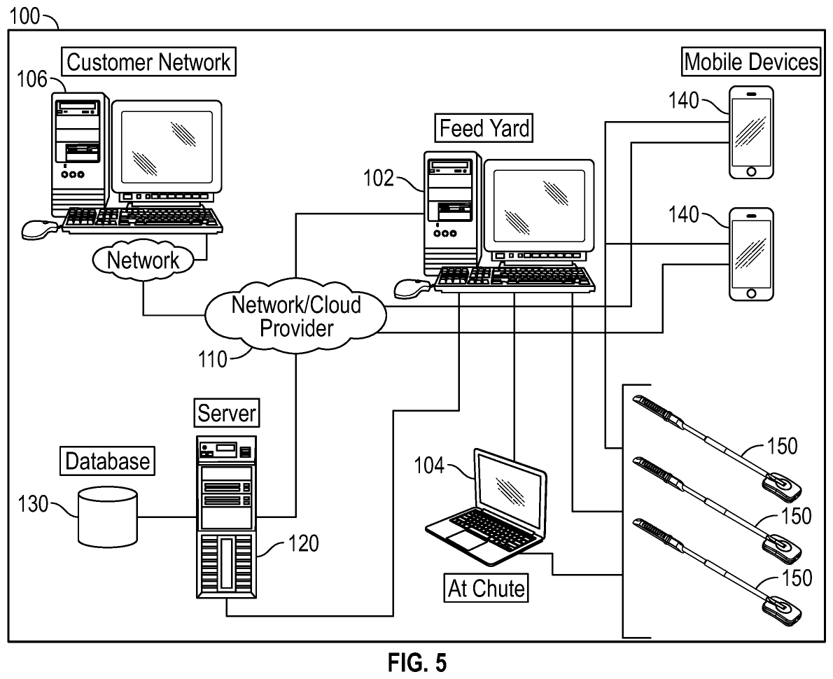

FIG. 5 provides an example computer processing and communication network that may be used in connection with the system and method of the invention disclosed herein. More specifically, FIG. 5 illustrates a block diagram of a system 100 that includes one or more user computers shown as feed yard computer 102, a chute side computer 104, and customer network 106, in which each of the computers 102, 104 and network 106 may alternatively comprise more than one computer. Specifically, the feed yard computer 102 represents one or more computers used in a feed yard or feedlot environment used to automatically control the accounting, feeding, and treatment of animals prior to harvesting. The chute side computer 104 represents one or more computers used in a feed lot environment that may be used to initially receive and record data regarding animals being received into the feedlot. The customer network 106 represents one or more computers of third parties who may seek to exchange data with the feed lot, such as financial institutions, cattle growers, and other third parties who are involved with a livestock industry. These user computers 102, 104, and network 106 may comprise general purpose personal computers (including, merely by way of example, personal computers and/or laptop computers running various versions of Microsoft's Windows® and/or Apple® operating systems) and/or workstation computers running any of a variety of commercially-available LINUX®, UNIX® or LINUX®-like operating systems. These user computers 102, 104, and network 106 may also have any of a variety of applications, including for example, database client and/or server applications, and web browser applications. Alternatively, the user computers 102, 104, and computers within network 106 may be any other electronic device, such as a thin-client computer, Internet-enabled mobile telephone, and/or personal digital assistant, capable of communicating via a network and/or displaying and navigating web pages or other types of electronic documents.

System 100 further includes a communications network 110. The network 110 may be any type of network familiar to those skilled in the art that can support data communications using any of a variety of commercially-available protocols, including without limitation TCP/IP, SNA, IPX, AppleTalk®, and the like. Merely by way of example, the communications network 110 maybe a local area network (“LAN”), such as an Ethernet network, a Token-Ring network and/or the like; a wide-area network; a virtual network, including without limitation a virtual private network (“VPN”); the Internet; an intranet; an extranet; a public switched telephone network (“PSTN”); an infra-red network; a wireless network (e.g., a network operating under any of the IEEE 802.11 suite of protocols, the Bluetooth™ protocol known in the art, and/or any other wireless protocol); and/or any combination of these and/or other networks.

The system may also include one or more server computers 120. One type of server may include a web server used to process requests for web pages or other electronic documents from user computers 102, 104, and network 106. The web server can be running an operating system including any of those discussed above, as well as any commercially-available server operating systems. The web server can also run a variety of server applications, including HTTP servers, FTP servers, CGI servers, database servers, Java servers, and the like. In some instances, the web server may publish operations available as one or more web services.

The system 100 may also include one or more file and/or application servers, which can, in addition to an operating system, include one or more applications accessible by a client running on one or more of the user computers 102, 104, and network 106. The file/application server(s) may be one or more general purpose computers capable of executing programs or scripts in response to the user computers 102, 104, and network 106. As one example, the server may execute one or more web applications. The web application may be implemented as one or more scripts or programs written in any programming language, such as Java®, C, C#™ or C++, and/or any scripting language, such as Perl, Python, or TCL, as well as combinations of any programming/scripting languages. The application server(s) may also include database servers, including without limitation those commercially available from Oracle®, Microsoft, Sybase®, IBM® and the like, which can process requests from database clients running on a user computer.

In one functional aspect, an application server may create web pages dynamically for displaying the functionality associated with the system to include the user interface of FIGS. 1 and 2. The web pages created by the web application server may be forwarded to a user computer via a web server. Similarly, the web server may be able to receive web page requests, web services invocations, and/or input data from a user computer and can forward the web page requests and/or input data to the web application server. In another functional aspect, the server 120 may also function as a file server.

The system 100 may also include a database 130. The database 130 may reside in a variety of locations. By way of example, database 130 may reside on a storage medium local to (and/or resident in) one or more of the computers 102, 104, and network 106. Alternatively, it may be remote from any or all of the computers 102, 104, and network 106, and in communication (e.g., via the network 110) with one or more of these. In a particular set of embodiments, the database 130 may reside in a storage-area network (“SAN”). Similarly, any necessary files for performing the functions attributed to the computers 102, 104, and network 106 may be stored locally on the respective computer and/or remotely, as appropriate. The database 130 may be a relational database, such as Oracle® database, that is adapted to store, update, and retrieve data in response to SQL-formatted commands.

The system may further include one or more mobile devices 140 such as “smart phones”. These mobile devices 140 communicate with the network 110 as by a web interface. The network 110 may also represent a cloud provider who facilitates communication with communication endpoints or computers of the customer network 106. The mobile devices 140 may communicate with any other of the computers in the system through the network 110, such as the feed yard computer system 102 as also shown in FIG. 5. The mobile devices have their own internal computer processing capabilities with integral computer processors and other supporting hardware and software. The mobile devices may be specially configured to run a mobile software application(s) in order to view user interfaces and to view and update system data. All of the functionality associated with the system as applied to the computers 102, 104, and 106 may be incorporated in the mobile devices 140 as modified by mobile software applications especially adapted for the mobile device hardware and operating systems. In connection with operating systems, it should therefore be understood that the mobile devices are not limited to any particular operating system, Apple iOS and Android-based systems being two examples.

FIG. 5 also schematically illustrates a plurality of measuring digital audio recording units 150 in which each may have supporting sensor circuitry (not shown) for recording and transmitting audio signals to the chute side computer or other computers within the network. For example, the digital audio recording units 150 could be the audio digital recording unit 60 shown in FIG. 2, as depicted.

In accordance with any of the computers 102, 104, and 106, these may be generally described as general-purpose computers with elements that cooperate to achieve multiple functions normally associated with general purpose computers. For example, the hardware elements 102, 104, and 106 may further include one or more input devices (e.g., a mouse, a keyboard, etc.); and one or more output devices (e.g., a display device, a printer, etc.). The computers may also include one or more storage devices. By way of example, storage device(s) may be disk drives, optical storage devices, solid-state storage device such as a random access memory (“RAM”) and/or a read-only memory (“ROM”), which can be programmable, flash-updateable and/or the like.

Each of the computers and server described herein may include a computer-readable storage media reader; a communications peripheral (e.g., a modem, a network card (wireless or wired), an infra-red communication device, etc.); working memory, which may include RAM and ROM devices as described above. The server may also include a processing acceleration unit, which can include a DSP, a special-purpose processor and/or the like.

The computer-readable storage media reader can further be connected to a computer-readable storage medium, together (and, optionally, in combination with storage device(s)) comprehensively representing remote, local, fixed, and/or removable storage devices plus storage media for temporarily and/or more permanently containing computer-readable information. The computers and serve permit data to be exchanged with the network 110 and/or any other computer, server, or mobile device.

The computers and server also comprise various software elements and an operating system and/or other programmable code such as program code implementing a web service connector or components of a web service connector. It should be appreciated that alternate embodiments of a computer may have numerous variations from that described above. For example, customized hardware might also be used and/or particular elements might be implemented in hardware, software (including portable software, such as applets), or both. Further, connection to other computing devices such as network input/output devices may be employed.

It should also be appreciated that the method described herein may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other type of optical disks, ROMs, RAMs, EPROMs, EEPROMs, magnetic or optical cards, flash memory, or other types of machine-readable mediums suitable for storing electronic instructions. Alternatively, the methods may be performed by a combination of hardware and software.

The term “software” as used herein shall be broadly interpreted to include all information processed by a computer processor, a microcontroller, or processed by related computer executed programs communicating with the software. Software therefore includes computer programs, libraries, and related non-executable data, such as online documentation or digital media. Executable code makes up definable parts of the software and is embodied in machine language instructions readable by a corresponding data processor such as a central processing unit of the computer. The software may be written in any known programming language in which a selected programming language is translated to machine language by a compile, interpreter or assembler element of the associated computer.

Considering the foregoing exemplary computer and communications network and elements described therein, In connection with one embodiment of the invention, it may be considered a software program or software platform with computer coded instructions that enable execution of the functionality associated with the user interface of FIGS. 1 and 2. More specifically, the invention may be considered a software program or software platform that executes the algorithm based on data inputs to the algorithm as described including, without limitation, heart and lung sound data, body weight, and rectal temperature.

In connection with another embodiment of the invention, it may be considered a combined software and hardware system including (a) a software program or software platform with computer coded instructions that enable execution of the functionality associated with the user interfaces of FIGS. 1 and 2 along with the execution of the algorithm to generate the treat/don't treat decision, and (b) hardware elements including the plurality of audio sensors that record auscultated sounds, a weigh scale for recording animal weight, and a rectal thermometer for recording animal temperature.

In connection with yet another embodiment of the invention, it may be considered a sub-combination including one or more user interfaces generated by the software and the field devices that provide inputs to a data processor of a computer that runs the software for purposes of generating the treatment decision by use of the algorithm.

While the invention is described herein with respect to multiple preferred embodiments, it should be understood that the invention is not strictly limited to these embodiments and therefore, the invention in totality should be considered commensurate with the scope of the claims appended hereto.

What is claimed is:

1. A system for determining a health status of an animal upon arrival at a location, comprising:
a computer processor for receiving and storing biometric data;
at least one recording device for capturing and recording biometric data from the animal, said device communicating with said computer processor, said device including at least an audio recorder that records the biometric data obtained by auscultated heart and lung sounds of the animal;
computer coded instructions executed by said computer processor including at least one algorithm for determining whether the animal should receive treatment;
a user interface associated with the computer processor for displaying information obtained by execution of said computer processor; and
wherein said algorithm includes a gradient boosted tree algorithm comprising input variables obtained from the recorded biometric data and corresponding to the recorded heart and lung sounds.

2. The system of claim 1, wherein:
said input variables further include at least one of a body weight of the animal and a rectal temperature of the animal.

3. The system of claim 1, wherein:
said algorithm incorporates the biometric data recorded as converted to numerical form for inputs to the algorithm, and in which raw signal dated recorded of the auscultated heart and lung sounds undergo bandpass filtering.

4. The system of claim 3, wherein:
said input variables include a heart rate.

5. A system, as claimed in claim 3, wherein said input variables include a respiration rate.

6. The system of claim 3, wherein said algorithm includes additional inputs including arithmetic expressions of the recorded biometric data, said expressions including at least one of a cardiopulmonary ratio, a cardiac breath gap, a cardiac index, and a cardiac max.

7. The system of claim 1, further including:
another user interface associated with the computer processor for displaying a health status of the animal, said health status including a likelihood the animal may develop BRD.

8. The system of claim 1, wherein:
said displayed information includes a user treatment decision for the animal.

9. The system of claim 8, wherein:
said at least one algorithm is a gradient boosted tree algorithm expressed by a function
F₀(x)=arg min γ Σᵢ₌₁ⁿL(yᵢ, γ);
wherein the function classifies the treatment decision to treat or not treat an animal;
wherein γ represents a specific combination of inputs that arrive at the treatment decision; and
wherein Σ L represents a misclassification error or number of falsely estimated instances i in a training set when compared to a true label yᵢ.

10. The system of claim 9, wherein:
said specific combination of inputs γ includes at least one of:
(a) a cardio-pulmonary ratio (cpr), calculated as
$$( \frac{hr percentile}{rr percentile} ) ,$$
said cpr ratio representing a magnitude of stress experienced by the animal upon arrival at the location;
(b) a cardiac breath gap (cbg), calculated as (heart rate−respiration rate);
(c) a cardiac index (ci), calculated as
$$( \frac{hr percentile}{bw percentile} ) ,$$
said ci index representing a relationship between heart performance and size of the animal; and
(d) a cardiac max (cm), calculated as
$$( \frac{h r}{75 th percentile hr ( based on bw and rt )} ) ,$$
said cm representing an estimation of how efficiently the animal is consuming oxygen upon arrival at the location.

11. The system of claim 9, wherein:
execution of said at least one algorithm by said computer processor includes a plurality of decision stumps and a generated outcome resulting in a treatment decision.

12. The system of claim 11, wherein:
said treatment decision is a treat decision.

13. The system of claim 11, wherein:
said treatment decision is a don't treat decision.

14. The system of claim 9, wherein:
execution of said at least one algorithm includes conducting iterative computations to arrive at the treatment decision.

15. A method for determining a health status of an animal upon arrival at a location, comprising:
providing a computer processor for receiving and storing biometric data;
providing at least one recording device;
recording raw biometric data from the animal by use of at least an audio recorder that records heart and lung sounds;
converting the raw biometric data recorded to manipulated heart and lung sound data;
providing computer coded instructions executed by the computer processor including at least one algorithm for determining a health status of the animal;
executing the algorithm with input variables corresponding to the manipulated heart and lung sound data;
generating a user interface associated with the computer processor for displaying to a user a health status of the animal; and
wherein said algorithm includes a gradient boosted tree algorithm comprising input variables corresponding to the manipulated heart and lung sound data of the animal.

16. The method of claim 15, wherein:
said at least one algorithm further includes computer coded instructions for determining whether the animal should receive treatment; and
said user interface further includes a treatment decision for the animal.

17. The method of claim 15, wherein:
said input variables further include at least one of a body weight of the animal and a rectal temperature of the animal.

18. The method of claim 15, wherein:
said manipulated heart and lung sound data includes the raw biometric data that undergoes bandpass filtering.

19. The method of claim 15, wherein:
said manipulated heart and lung sound data includes a recorded heart rate.

20. The method of claim 15, wherein:
said manipulated heart and lung sound data includes a respiration rate and selected recorded frequencies and amplitudes of recorded lung sounds.

21. The method of claim15, wherein said algorithm includes additional inputs including arithmetic expressions of recorded heart and lung sound data, said expressions including at least one of a cardiopulmonary ratio, a cardiac breath gap, a cardiac index, and a cardiac max.

22. The method of claim15, further including:
generating a user interface associated with the computer processor wherein said health status including a likelihood the animal may develop BRD.

23. The method of claim 15, wherein:
said health status includes a user treatment decision for the animal.

24. The method of claim 15, wherein:
said at least one algorithm is a gradient boosted tree algorithm expressed by a function
F₀(x)=arg min γ Σᵢ₌₁ⁿL(yᵢ, γ);
wherein the function classifies the treatment decision to treat or not treat an animal;
wherein γ represents a specific combination of inputs that arrive at the treatment decision; and
wherein Σ L represents a misclassification error or number of falsely estimated instances i in a training set when compared to a true label yᵢ.

25. The method of claim 24, wherein:
said specific combination of inputs γ includes at least one of:
(a) a cardio-pulmonary ratio (cpr), calculated as
$$( \frac{hr percentile}{rr percentile} ) ,$$
said cpr ratio representing a magnitude of stress experienced by the animal upon arrival at the location;
(b) a cardiac breath gap (cbg), calculated as (heart rate−respiration rate);
(c) a cardiac index (ci), calculated as
$$( \frac{hr percentile}{bw percentile} ) ,$$
said ci index representing a relationship between heart performance and size of the animal; and
(d) a cardiac max (cm), calculated as
$$( \frac{h r}{75 t h p e r c e n t i l e h r ( b a s e d o n b w a n d r t )} )$$
said cm representing an estimation of how efficiently the animal is consuming oxygen upon arrival at the location.

26. The method of claim 24, wherein:
execution of said at least one algorithm by said computer processor includes a plurality of decision stumps and a generated outcome resulting in a treatment decision.

27. The method of claim 26, wherein:
said treatment decision is a treat decision.

28. The method of claim 26, wherein:
said treatment decision is a don't treat decision.

29. The method of claim 24, wherein:
execution of said at least one algorithm includes conducting iterative computations to arrive at the treatment decision.

30. A non-transitory computer-readable medium containing computer executable instructions, wherein, when executed by a computer processor, the instructions cause the computer processor to execute a method to determine a health status of an animal the computer-readable instructions comprising:
instructions to receive and store data corresponding to recorded heart and lung sounds;
instructions to convert the recorded heart and lung sounds to manipulated heart and lung sound data;
instructions to execute an algorithm to determine the health status of the animal, wherein the algorithm comprises input variables corresponding to the manipulated heart and lung sound data;
instructions to generate a user interface associated with the computer processor for displaying to a user the health status of the animal, wherein said health status includes technical recommendations for further evaluating whether the health status requires further analysis; and
wherein said algorithm includes a gradient boosted tree algorithm comprising input variables corresponding to the manipulated heart and lung sound data of the animal.

31. The non-transitory computer-readable medium of claim 30 wherein:
said instructions to execute the algorithm further include instructions to determine whether the animal should receive treatment; and
said instructions to generate the user interface further include instructions to display to the user a treatment decision for the animal.

32. The non-transitory computer-readable medium of claim 30, wherein:
said health status includes a user treatment decision for the animal.

33. The non-transitory computer-readable medium of claim 31, wherein:
said at least one algorithm is a gradient boosted tree algorithm expressed by a function
F₀(x)=arg min γ Σᵢ₌₁ⁿL(Yᵢ, γ);
wherein the function classifies the treatment decision to treat or not treat an animal;
wherein γ represents a specific combination of inputs that arrive at the treatment decision; and
wherein Σ L represents a misclassification error or number of falsely estimated instances i in a training set when compared to a true label yᵢ).

34. The non-transitory computer-readable medium of claim 33, wherein:
said specific combination of inputs γ includes at least one of:
(a) a cardio-pulmonary ratio (cpr), calculated as
$$( \frac{hr percentile}{rr percentile} ) ,$$
said cpr ratio representing a magnitude of stress experienced by the animal upon arrival at the location;
(b) a cardiac breath gap (cbg), calculated as (heart rate−respiration rate);
(c) a cardiac index (ci), calculated as
$$( \frac{hr percentile}{bw percentile} ) ,$$
said ci index representing a relationship between heart performance and size of the animal; and
(d) a cardiac max (cm), calculated as
$$( \frac{h r}{75 t h p e r c e n t i l e h r ( b a s e d o n b w a n d r t )} )$$
said cm representing an estimation of how efficiently the animal is consuming oxygen upon arrival at the location.

35. The non-transitory computer-readable medium of claim 33, wherein:
execution of said at least one algorithm by said computer processor includes a plurality of decision stumps and a generated outcome resulting in a treatment decision.

36. The non-transitory computer-readable medium of claim 35, wherein:
said treatment decision is a treat decision.

37. The non-transitory computer-readable medium of claim 35, wherein:
said treatment decision is a don't treat decision.

38. The non-transitory computer-readable medium of claim 33, wherein:
execution of said at least one algorithm includes conducting iterative computations to arrive at the treatment decision.
